(19) United States
(12) Patent Application Publication
(10) Pub. No.: US 2026/0207662 A1
(43) Pub. Date: Jul. 23, 2026

(54) Thickened Silver Diamine Fluoride Composition For Treatment of Gingivitis

(71) Applicant: ADVANTAGE SILVER DENTAL ARREST, LLC, Salem, OR (US)

(72) Inventor: Peter M. MILGROM, Seattle, WA (US)

(73) Assignee: ADVANTAGE SILVER DENTAL ARREST, LLC, Salem, OR (US)

(21) Appl. No.: 19/143,512

(22) PCT Filed: Jan. 9, 2023
(86) PCT No.: PCT/US2023/060305

§ 371 Date: Jun. 26, 2025

(51) Int. Cl. A61K 33/38; A61K 33/16; A61P 1/02

(52) U.S. Cl. CPC A61K 33/38; A61K 33/16; A61P 1/02

(57) ABSTRACT

A method of treating gingivitis in a subject by administering a therapeutically effective amount of a thickened silver diamine fluoride composition to a tooth, wherein said silver diamine fluoride composition comprises silver diamine fluoride and has at least one of the following features: (A) the silver diamine fluoride composition is thixotropic and has a viscosity of 0.02 to 500 Pa·s, as measured by a TA AR-G2 rheometer with a 40 mm cone and plate, and carried out at a temperature of about 23° C., and a shear rate of from 0.1/second to 100/second; and (B) the silver diamine fluoride composition comprises a thickening agent, wherein the thickening agent is in an amount of 0.1 wt. % to 15 wt. %, based on a total weight of the composition.

### TECHNICAL FIELD

The present disclosure relates to a method for treating gingivitis in a subject, the method comprising administering a therapeutically effective amount of a thickened silver diamine fluoride composition to a tooth, said composition having a consistency to allow a dentist to control the application of the thickened silver diamine fluoride composition.

### BACKGROUND OF THE INVENTION

Humans as well as their pets, such as dogs and cats, are subject to dental health problems. These problems can be traced to the formation of bacterial plaque that forms on the exterior surface of teeth. Plaque is a gelatinous mass; a sticky film of bacteria, polysaccharides and salivary proteins that are not easily washed away. Plaque is now generally recognized as the main culprit in poor oral health.

Gingivitis is an inflammatory disease of the gums, which occurs on the gingiva (gums) of the oral cavity most commonly on the soft tissue area around the tooth border and can eventually lead to periodontitis. The main cause is usually dysbiosis of the microbial community in the subgingival space, where the bacteria induce a dysregulated and destructive inflammatory response in the host. Periodontitis can eventually lead to tooth loss, through the continuous inflammation-mediated tissue damage surrounding the teeth, as well as resorption of the supporting alveolar bone. Gingivitis results from an accumulation of plaque on the teeth. Bacteria in plaque invade the spaces between teeth and gums and begin producing toxins that cause gum irritation, swelling, and bleeding. Daily flossing and twice daily brushing of the teeth are traditionally recommended as prophylaxis and home treatment for gingivitis. These traditional treatments can be problematic for individuals suffering from gingivitis who fear blood as flossing and vigorous brushing often cause bleeding of inflamed gums. These traditional treatments can also be problematic as many individuals lack the time, discipline, or technique to maintain a flossing and brushing regimen sufficient to prevent and/or treat gingivitis without the help of additional treatments that specifically target the gingivitis causing bacteria.

Silver diamine fluoride was approved for marketing in the United States in August 2014 by the United States Food and Drug Administration for intraoral human treatment of dentin tooth hypersensitivity. Liquid silver diamine fluoride has also been used in the treatment of gingivitis. Clinicians have reported reductions in gingivitis when the liquid silver diamine fluoride in patients with special health care needs and researchers have demonstrated a reduction of gingivitis in a randomized clinical trial in older adults. Laboratory investigators have demonstrated a reduction in the oral bacteria associated with gingivitis and periodontitis in these individuals. See Lim GXD, Yang J. Effect of silver diamine fluoride on hyperplastic gingivitis in an adult with intellectual disability-A case report. Spec Care Dentist. 2022 January; 42(1): 73-79. doi: 10.1111/scd.12633. Epub 2021 Jul. 22. PMID: 34293215; Chu C H, Lee A H, Zheng L, Mei M L, Chan G C. Arresting rampant dental caries with silver diamine fluoride in a young teenager suffering from chronic oral graft versus host disease post-bone marrow transplantation: a case report. BMC Res Notes. 2014 Jan. 3; 7:3. doi: 10.1186/1756-0500-7-3. PMID: 24383434; PMCID: PMC3880541; Rams T E, Sautter J D, Ramírez-Martínez GJ, Whitaker E J. Antimicrobial activity of silver diamine fluoride on human periodontitis microbiota. Gen Dent. 2020 September-October; 68 (5): 24-28. PMID: 32857044; and Alsherhri W et al. Efficacy of 38% Silver Diamine Fluoride to Treat Gingivitis: A Randomized Clinical Trial. Int J Dent Hug, 2022, in press.

The commercially available 38% silver diamine fluoride product in the United States is comprised of 25% silver, 5% fluoride, and 8% ammonia as a stabilizing agent. However, silver diamine fluoride containing ammonia has a low viscosity, and thus is a liquid at room temperature. Therefore, the application of this material must be carried out carefully as it is difficult to control the intended location of the liquid on the tooth at the time of application. If the liquid runs off the intended location on the tooth, it may come in contact with gums, other mucosal surfaces, tongue, lips, and face. Furthermore, the silver diamine fluoride (SDF) may result in the formation of silver oxide which may result in a pigmentation of the applied area. This may result in a range of brown to dark brown discoloration which is undesirable on certain affected areas, including the skin.

Accordingly, what is needed is an SDF composition that has an improved viscosity which allows for a dentist to better control the application of the formulation to the intended area on the teeth as a topical composition used in the treatment of gingivitis without inadvertent extension to other oral structures or spills on the face or equipment.

Additional features of the present disclosure will become apparent to those skilled in the art upon consideration of the following detailed description of the presently perceived best mode of carrying out the disclosure.

### SUMMARY AND TERMS

The inventor has surprisingly found that adding a thickening agent to the silver diamine fluoride (SDF) can meet these needs for the treatment of gingivitis in a subject with no evidence of unintended adverse effects, while maintaining the desirable properties of the SDF.

In a first aspect, the disclosure relates to a method of treating gingivitis in a subject, the method comprising administering a therapeutically effective amount of a silver diamine fluoride composition to a surface of one or more teeth but not directly to the gums, wherein said silver diamine fluoride composition comprises silver diamine fluoride and has at least one of the following features:
(A) the silver diamine fluoride composition is thixotropic and has a viscosity of 0.02 Pa·s to 500 Pa·s as measured by a TA AR-G2 rheometer with a 40 mm cone and plate, and carried out at a temperature of about 23° C., and a shear rate of from 0.1/second to 100/second; and
(B) the silver diamine fluoride composition comprises a thickening agent, wherein the thickening agent is in an amount of 0.1 wt. % to 15 wt. %, based on a total weight of the composition.

2. The method of sentence 1, wherein the silver diamine fluoride may be in a concentration of from 10 wt. % to 40 wt. %, or 12 wt. % to 38 wt. % or 15 wt. % to 35 wt. % based on the total weight of the composition, preferably, the silver diamine fluoride may be present in a concentration of about 38 wt. %, based on the total weight of the composition.

3. The method of sentence 1 or 2, which may further comprise a thickening agent.

4. The method of sentence 3, wherein the thickening agent may be in an amount of 0.1 wt. % to 15 wt. %, or 0.25 to 10 wt. %, or 0.5 to 5 wt. %, based on the total weight of the composition.

5. The method of any one of sentences 3-4, wherein the thickening agent may be a cellulose derivative (“cellulose gum”) such as carboxymethyl cellulose (CMC), methyl cellulose, hydroxyethyl cellulose, hydroxypropyl methyl cellulose, microcrystalline cellulose, and mixtures thereof; polyvinyl pyrrolidone; xanthan; an admixture of glycerin and polyacrylate; carrageenans such as iota-carrageenan, kappa-carrageenan, kappa2-carrageenan, lambda-carrageenan, and mixtures thereof; guar gum; gum karaya; gum arabic; gum tragacanth; and mixtures thereof, hydrated silica and colloidal silica may be used as thickeners, or silica thickeners.

6. The method of any one of sentences 3-5, wherein the thickening agent may be selected from carboxy methyl cellulose (CMC), microcrystalline cellulose, and an admixture of glycerin and polyacrylate, and more preferably, carboxy methyl cellulose.

7. The method of any one of sentences 3-6, wherein the silver diamine fluoride composition may be prepared by mixing silver diamine fluoride with the thickening agent, wherein the silver diamine fluoride may be in an amount of about 38 wt. % and the thickening agent may be carboxy methyl cellulose (CMC) in an amount of 0.1 wt. % to 5 wt. % based on the total weight of the composition.

8. The method of any one of sentences 1-7, wherein the silver diamine fluoride composition may further comprise a clathrate of glyceryl acrylate and glyceryl polyacrylate that encloses water molecules, or a cyclodextrin, wherein the cyclodextrin may preferably be an α-cyclodextrin, β-cyclodextrin, and γ-cyclodextrin, and derivatives of cyclodextrin such as methylated cyclodextrins, ethylated cyclodextrins, hydroxypropyl cyclodextrins, or hydroxyethyl cyclodextrins.

9. The method of sentence 8, wherein the clathrate may be present in an amount of from 0.1 wt. % to 15 wt. %, or 0.25 wt. % to 10 wt. %, or 0.5 wt. % to 5 wt. %, based on the total weight of the composition.

10. The method of any one of sentences 1-9, wherein the subject may be a mammal, preferably a cat, a dog, or a human, more preferably a human.

11. The method of any one of sentences 1-10, wherein the silver diamine fluoride composition may further comprise a coloring agent.

12. The method of sentence 11, wherein the coloring agent may be triarylmethane dye.

13. The method of any one of sentences 1-12, wherein the silver diamine fluoride composition may further comprise a flavoring agent.

14. The method of any one of sentences 1-13, wherein the composition may be a thixotropic fluid that can undergo an isothermal gel-sol-gel transformation.

15. The method of any one of sentences 1-14, wherein the composition may have a pH of from 7.0 to about 13.5, or from about 10.0 to about 10.5.

16. The method of any one of sentences 1-15, wherein the viscosity may be from 0.1 Pa·s to 500 Pa's, or from 0.1 Pa's to 450 Pa·s, and a shear rate of from 0.1/second to 100/second; or greater than 75 Pa·s, or greater than 100 Pa·s, or greater than 125 Pa·s at a shear rate of 0.1/sec and less than 15 Pa's, or less than 10 Pa's, or less than 8 Pa's at a shear rate of 100/sec, as measured by a TA AR-G2 rheometer with a 40 mm cone and plate, and carried out at a temperature of about 23° C.

17. The method of any one of sentences 1-16, wherein the silver diamine fluoride may provide 1,000 ppm to 75,000 ppm fluoride to the silver diamine fluoride composition, preferably 10,000 to 60,000 ppm fluoride to the silver diamine fluoride composition, more preferably 40,000 to 55,000 ppm fluoride to the silver diamine fluoride composition, based on the total weight of the composition; or the silver diamine fluoride may provide 100 ppm to 14,000 ppm silver to the silver diamine fluoride composition, preferably 1000 to 12,000 ppm silver to the silver diamine fluoride composition, more preferably 4,000 to 9,000 ppm silver to the silver diamine fluoride composition, based on the total weight of the composition.

18. In a second aspect, the present invention relates to a method of inhibiting, reducing or preventing growth or biofilm formation of gingivitis-causing bacteria in an oral cavity of a subject suffering from or susceptible to gingivitis comprising topically contacting a silver diamine fluoride composition with a surface of one or more teeth of the subject without directly contacting the gums, wherein the silver diamine fluoride composition comprises silver diamine fluoride and has at least one of the following features:
(A) the silver diamine fluoride composition is thixotropic and has a viscosity of 0.02 Pa's to 500 Pa·s, as measured by a TA AR-G2 rheometer with a 40 mm cone and plate, and carried out at a temperature of about 23° C., and a shear rate of from 0.1/second to 100/second; and
(B) the silver diamine fluoride composition comprises a thickening agent, wherein the thickening agent is in an amount of 0.1 wt. % to 15 wt. %, based on a total weight of the composition.

19. The method of sentence 18, wherein the silver diamine fluoride may be in a concentration of from 10 wt. % to 40 wt. %, or 12 wt. % to 38 wt. % or 15 wt. % to 35 wt. % based on the total weight of the composition, preferably, the silver diamine fluoride may be present in a concentration of about 38 wt. %, based on the total weight of the composition.

20. The method of sentence 18 or 19, wherein the silver diamine fluoride composition may further comprise a thickening agent.

21. The method of sentence 20, wherein the thickening agent may be in an amount of 0.1 wt. % to 15 wt. %, or 0.25 wt. % to 10 wt. %, or 0.5 wt. % to 5 wt. %, based on the total weight of the composition.

22. The method of any one of sentences 20-21, wherein the thickening agent may be a cellulose derivative (“cellulose gum”) such as carboxymethyl cellulose (CMC), methyl cellulose, hydroxyethyl cellulose, hydroxypropyl methyl cellulose, microcrystalline cellulose, and mixtures thereof; polyvinyl pyrrolidone; xanthan; an admixture of glycerin and polyacrylate; carrageenans such as iota-carrageenan, kappa-carrageenan, kappa2-carrageenan, lambda-carrageenan, and mixtures thereof; guar gum; gum karaya; gum arabic; gum tragacanth; and mixtures thereof, hydrated silica and colloidal silica may be used as thickeners, or silica thickeners.

23. The method of any one of sentences 20-22, wherein the thickening agent may be selected from carboxy methyl cellulose (CMC), microcrystalline cellulose, and an admixture of glycerin and polyacrylate, and more preferably, carboxy methyl cellulose.

24. The method of any one of sentences 20-23, wherein the silver diamine fluoride composition may be prepared by mixing silver diamine fluoride with the thickening agent, wherein the silver diamine fluoride may be in an amount of about 38 wt. % and the thickening agent may be carboxy methyl cellulose (CMC) in an amount of 0.1 wt. % to 5 wt. % based on the total weight of the composition.

25. The method of any one of sentences 18-24, wherein the silver diamine fluoride composition may further comprise a clathrate of glyceryl acrylate and glyceryl polyacrylate that encloses water molecules, or a cyclodextrin, wherein the cyclodextrin is preferably α-cyclodextrin, β-cyclodextrin, and γ-cyclodextrin, and derivatives of cyclodextrin such as methylated cyclodextrins, ethylated cyclodextrins, hydroxypropyl cyclodextrins, or hydroxyethyl cyclodextrins.

26. The method of sentences 25, wherein the clathrate may be present in an amount of from 0.1 wt. % to 15 wt. %, or 0.25 wt. % to 10 wt. %, or 0.5 to 5 wt. %, based on the total weight of the composition.

27. The method of any one of sentences 18-26, wherein the subject may be a mammal, preferably a cat, a dog, or a human, more preferably a human.

28. The method of any one of sentences 18-27, wherein the silver diamine fluoride composition may further comprise a coloring agent.

29. The method of sentence 28, wherein the coloring agent may be triarylmethane dye.

30. The method of any one of sentences 18-29, wherein the silver diamine fluoride composition may further comprise a flavoring agent.

31. The method of any one of sentences 18-30, wherein the composition may be a thixotropic fluid that can undergo an isothermal gel-sol-gel transformation.

32. The method of any one of sentences 18-31, wherein the composition may have a pH of from 7.0 to about 13.5, or from about 10.0 to about 10.5.

33. The method of any one of sentences 18-32, wherein the viscosity may be from 0.1 Pa's to 500 Pa·s, or from 0.1 Pa's to 450 Pa·s, and a shear rate of from 0.1/second to 100/second; or greater than 75 Pa's, or greater than 100 Pa's, or greater than 125 Pa·s at a shear rate of 0.1/sec and less than 15 Pa·s, or less than 10 Pa's, or less than 8 Pa's at a shear rate of 100/sec, as measured by a TA AR-G2 rheometer with a 40 mm cone and plate, and carried out at a temperature of about 23° C.

34. The method of any one of sentences 18-33, wherein the silver diamine fluoride may provide 1,000 ppm to 75,000 ppm fluoride to the silver diamine fluoride composition, preferably 10,000 to 60,000 ppm fluoride to the silver diamine fluoride composition, more preferably 40,000 to 55,000 ppm fluoride to the silver diamine fluoride composition, based on the total weight of the composition; or the silver diamine fluoride may provide 100 ppm to 14,000 ppm silver to the silver diamine fluoride composition, preferably 1000 to 12,000 ppm silver to the silver diamine fluoride composition, more preferably 4,000 to 9,000 ppm silver to the silver diamine fluoride composition, based on the total weight of the composition.

35. In another aspect, the present invention relates to a use of a silver diamine fluoride composition in a method of treating gingivitis in a subject, the method comprising administering a therapeutically effective amount of a silver diamine fluoride composition to a surface of one or more teeth but not directly to the gums, wherein said silver diamine fluoride composition comprises silver diamine fluoride and has at least one of the following features:
(A) the silver diamine fluoride composition is thixotropic and has a viscosity of 0.02 Pa's to 500 Pa·s, as measured by a TA AR-G2 rheometer with a 40 mm cone and plate, and carried out at a temperature of about 23° C., and a shear rate of from 0.1/second to 100/second; and
(B) the silver diamine fluoride composition comprises a thickening agent, wherein the thickening agent is in an amount of 0.1 wt. % to 15 wt. %, based on a total weight of the composition.

36. The use of sentence 35, wherein the silver diamine fluoride may be in a concentration of from 10 wt. % to 40 wt. %, or 12 wt. % to 38 wt. % or 15 wt. % to 35 wt. % based on the total weight of the composition, preferably, the silver diamine fluoride may be present in a concentration of about 38 wt. %, based on the total weight of the composition.

37. The use of sentence 35 or 36, wherein the silver diamine fluoride composition may further comprise a thickening agent.

38. The use of sentence 37, wherein the thickening agent may be in an amount of 0.1 wt. % to 15 wt. %, or 0.25 wt. % to 10 wt. %, or 0.5 wt. % to 5 wt. %, based on the total weight of the composition.

39. The use of any one of sentences 37-38, wherein the thickening agent may be a cellulose derivative (“cellulose gum”) such as carboxymethyl cellulose (CMC), methyl cellulose, hydroxyethyl cellulose, hydroxypropyl methyl cellulose, microcrystalline cellulose, and mixtures thereof; polyvinyl pyrrolidone; xanthan; an admixture of glycerin and polyacrylate; carrageenans such as iota-carrageenan, kappa-carrageenan, kappa2-carrageenan, lambda-carrageenan, and mixtures thereof; guar gum; gum karaya; gum arabic; gum tragacanth; and mixtures thereof, hydrated silica and colloidal silica may be used as thickeners, or silica thickeners.

40. The use of any one of sentences 37-39, wherein the thickening agent may be selected from carboxy methyl cellulose (CMC), microcrystalline cellulose, and an admixture of glycerin and polyacrylate, and more preferably, carboxy methyl cellulose.

41. The use of any one of sentences 37-40, wherein the silver diamine fluoride composition may be prepared by mixing silver diamine fluoride with the thickening agent, wherein the silver diamine fluoride may be in an amount of about 38 wt. % and the thickening agent may be carboxy methyl cellulose (CMC) in an amount of 0.1 wt. % to 5 wt. % based on the total weight of the composition.

42. The use of any one of sentences 35-41, wherein the silver diamine fluoride composition may further comprise a clathrate of glyceryl acrylate and glyceryl polyacrylate that encloses water molecules, or a cyclodextrin, wherein the cyclodextrin is preferably α-cyclodextrin, β-cyclodextrin, and γ-cyclodextrin, and derivatives of cyclodextrin such as methylated cyclodextrins, ethylated cyclodextrins, hydroxypropyl cyclodextrins, or hydroxyethyl cyclodextrins.

43. The use of sentence 42, wherein the clathrate may be present in an amount of from 0.1 wt. % to 15 wt. %, or 0.25 wt. % to 10 wt. %, or 0.5 wt. % to 5 wt. %, based on the total weight of the composition.

44. The use of any one of sentences 35-43, wherein the subject may be a mammal, preferably a cat, a dog, or a human, more preferably a human.

45. The use of any one of sentences 35-44, wherein the silver diamine fluoride composition may further comprise a coloring agent.

46. The use of sentence 45, wherein the coloring agent may be triarylmethane dye.

47. The use of any one of sentences 35-46, wherein the silver diamine fluoride composition may further comprise a flavoring agent.

48. The use of any one of sentences 35-47, wherein the silver diamine fluoride composition may be a thixotropic fluid that can undergo an isothermal gel-sol-gel transformation.

49. The use of any one of sentences 35-48, wherein the composition may have a pH of from 7.0 to about 13.5, or from about 10.0 to about 10.5.

50. The use of any one of sentences 35-49, wherein the viscosity may be from 0.1 Pa's to 500 Pa's, or from 0.1 Pa's to 450 Pa's, and a shear rate of from 0.1/second to 100/second; or greater than 75 Pa's, or greater than 100 Pa·s, or greater than 125 Pa·s at a shear rate of 0.1/sec and less than 15 Pa's, or less than 10 Pa's, or less than 8 Pa·s at a shear rate of 100/sec, as measured by a TA AR-G2 rheometer with a 40 mm cone and plate, and carried out at a temperature of about 23° C.

51. The use of any one of sentences 35-50, wherein the silver diamine fluoride may provide 1,000 ppm to 75,000 ppm fluoride to the silver diamine fluoride composition, preferably 10,000 to 60,000 ppm fluoride to the silver diamine fluoride composition, more preferably 40,000 to 55,000 ppm fluoride to the silver diamine fluoride composition, based on the total weight of the composition; or the silver diamine fluoride may provide 100 ppm to 14,000 ppm silver to the silver diamine fluoride composition, preferably 1000 to 12,000 ppm silver to the silver diamine fluoride composition, more preferably 4,000 to 9,000 ppm silver to the silver diamine fluoride composition, based on the total weight of the composition.

The following definitions of terms are provided in order to clarify the meanings of certain terms as used herein.

The terms “topical” and “topically” refer to application of a composition as disclosed herein to an outer surface of the teeth. If the tooth is coated with plaque, then the topical treatment includes treating the surface of the plaque. The surfaces of the teeth include the subgingival surface and the buccal surface. Also, the tooth may or may not have root surface tooth decay, and as such, the composition may be applied directly to the decayed portion of the tooth. There may be various ways to apply a topical paste or gel in accordance with the embodiments described herein. For example, the paste or gel may be applied to the teeth using a finger, cotton ball, or Q-tip. In some embodiments, the patient may let the paste or gel sit on the teeth for 1 to 30 minutes. In some embodiments, the patient may rinse the oral cavity with water after allowing the paste or gel to sit on the teeth. For example, while keeping the lips closed, the patient may make a swishing motion to move the water around the oral cavity.

The term “active agent” or “active ingredient” refers to a substance which has activity in preventing, reducing or inhibiting growth and/or biofilm formation of gingivitis-causing bacteria.

The presence, absence, or state of gingivitis can be determined by a health professional or lay person using methods that are known in the art. For example, gingivitis may be present when the patient is presenting with one or more of the following symptoms: swollen or puffy gums; dusky red or dark red gums; gums that bleed easily when brushed or flossed; bad breath; receding gums; and tender gums.

The present invention further includes treating gingivitis. “Treating” gingivitis refers to the prevention or cessation or reduction of progression of the symptoms discussed in the previous paragraph. For instance, treating gingivitis includes the prevention or cessation or reduction of progression of the plaque buildup in the subgingival space and/or swelling of the gums. Treating gingivitis includes preventing the plaque from beginning or getting worse.

As used herein, the term “effective amount” or “therapeutically effective amount” means an amount of a composition comprising an antimicrobial material, for example, SDF, that is effective to reverse a disease state, arrest a disease state, slow the progression of a disease state, ameliorate a disease state, relieves symptoms of a disease state or has any other beneficial consequences for the treated subject. For example, the compositions disclosed herein may have a topical therapeutic effect in inhibiting, reducing and/or preventing growth and/or biofilm formation of gingivitis-causing bacteria. This effect may decrease the likelihood that a subject may develop gingivitis in the oral cavity or slow the progression of gingivitis in the oral cavity of the subject.

As used herein, the term “about” refers to an approximately +/−10% variation from a given value. It is to be understood that such a variation is always included in any given value provided herein, whether or not it is specifically referred to.

The term “excipient” refers to an inactive substance that serves as a medium for the active agents and may be used in the production of a product comprising the composition to ensure the stability, efficacy, taste and appearance of the product is maintained from the time of production to when the product is consumed by the consumer.

A “disease” is a state of health of an animal wherein the subject cannot maintain homeostasis, and wherein if the disease is not ameliorated then the subject's health continues to deteriorate.

A “normal” subject does not have a clinical manifestation of gingivitis. In some instances a “normal” subject has a depth of gingival pocket of less than about 3 mm.

The terms “patient,” “subject,” “individual,” and the like are used interchangeably herein, and refer to any animal, or cells thereof whether in vitro or in situ, amenable to the methods described herein. In some non-limiting embodiments, the patient, subject or individual is mammal, such as a human, dog or cat, preferably a human.

### DETAILED DESCRIPTION OF THE INVENTION

In a first aspect, disclosed is a method of treating gingivitis in a subject, the method comprising administering a therapeutically effective amount of a silver diamine fluoride composition to a surface of a tooth but not directly to the gums, wherein said silver diamine fluoride composition comprises silver diamine fluoride and has at least one of the following features:
(A) the silver diamine fluoride composition is thixotropic and has a viscosity of 0.02 Pa·s to 500 Pa·s, or from 0.1 Pa·s to 500 Pa·s, or from 0.1 Pa·s to 450 Pa·s as measured by a TA AR-G2 rheometer with a 40 mm cone and plate, and carried out at a temperature of about 23° C., and a shear rate of from 0.1/second to 100/second; and
(B) the silver diamine fluoride composition comprises a thickening agent, wherein the thickening agent is in an amount of 0.1 wt. % to 15 wt. %, or 0.25 wt. % to 10 wt. % or 0.5 wt. % to 5 wt. %, based on a total weight of the composition.

In a second aspect of the disclosure, is a method of inhibiting, reducing or preventing growth or biofilm formation of gingivitis-causing bacteria in an oral cavity of a subject comprising topically contacting a silver diamine fluoride composition with a surface of a tooth of the subject without directly contacting the gums, wherein the silver diamine fluoride composition comprises silver diamine fluoride and has at least one of the following features:
(A) the silver diamine fluoride composition is thixotropic and has a viscosity of 0.02 Pa·s to 500 Pa·s, or from 0.1 Pa·s to 500 Pa·s, or from 0.1 Pa·s to 450 Pa's as measured by a TA AR-G2 rheometer with a 40 mm cone and plate, and carried out at a temperature of about 23° C., and a shear rate of from 0.1/second to 100/second; and
(B) the silver diamine fluoride composition comprises a thickening agent, wherein the thickening agent is in an amount of 0.1 wt. % to 15 wt. %, or 0.25 wt. % to 10 wt. % or 0.5 wt. % to 5 wt. %, based on a total weight of the composition.

It was found that adding a thickening agent to the silver diamine fluoride improves the ability of a dentist or user to control the application of the oral composition. In aspects of the present invention, there includes a step of administering a therapeutically effective amount of a silver diamine fluoride composition to a surface of a tooth but not directly to the gums, or a step of contacting a silver diamine fluoride composition with a surface of a tooth of the subject without directly contacting the gums. It is envisioned that there may be some incidental contact of the silver diamine fluoride composition to the gums, preferably less than 20 wt. % of the composition, more preferably less than 5 wt. % of the composition, and more preferably, less than 1 wt. % of the composition can incidentally contact the gums.

Silver diamine fluoride is an effective agent against bacteria that can be found in the microbial community in the subgingival space and buccal surfaces of the teeth. The main cause is usually dysbiosis of the microbial community in the subgingival space, where the bacteria induce a dysregulated and destructive inflammatory response in the host. The SDF is employed to kill the bacteria and arrest the progression of the inflammatory response. The fluoride causes the formation of calcium fluoride which serves as a fluoride reservoir to promote remineralization of lesions and prevent them forming in other teeth in the mouth.

The SDF compound is commercially available in the form of aqueous solutions with concentrations ranging from 10 wt. % to 40 wt. %, but preferably is present in a concentration of about 40 wt. %. The terms “silver diamine fluoride” or “silver diamine fluoride” or “diamine silver fluoride”, “AgF(NH₃)₂” may be used interchangeably.

The thickening agent can be present in an amount such that the composition for oral application may be thixotropic and have a viscosity of from 0.02 Pa·s to 500 Pa·s, or from 0.1 Pa·s to 500 Pa's, or from 0.1 Pa·s to 450 Pa·s, as measured by a TA AR-G2 rheometer with a 40 mm cone and plate, and carried out at room temperature (about 23° C.), and a shear rate of 0.1/second to 100/second. In some embodiments, the silver diamine fluoride composition may have a viscosity of greater than 75 Pa's, or greater than 100 Pa·s, or greater than 125 Pa·s at a shear rate of 0.1/sec and less than 15 Pa·s, or less than 10 Pa·s, or less than 8 Pa's at a shear rate of 100/sec. Suitable examples of thickening agents may be selected from cellulose derivative (“cellulose gum”) such as carboxymethyl cellulose (CMC), methyl cellulose, hydroxyethyl cellulose, hydroxypropyl methyl cellulose, microcrystalline cellulose, and mixtures thereof; polyvinyl pyrrolidone; xanthan; an admixture of glycerin and polyacrylate; carrageenans such as iota-carrageenan, kappa-carrageenan, kappa2-carrageenan, lambda-carrageenan, and mixtures thereof; guar gum; gum karaya; gum arabic; gum tragacanth; and mixtures thereof, hydrated silica and colloidal silica may be used as thickeners, or silica thickeners. Preferably, the silver diamine fluoride composition does not include poly(propylene oxide)/poly(ethylene oxide) copolymers or generally, poloxomers. Preferably, the thickening agent is a carboxymethyl cellulose. The thickening agent may be present in an amount of 0.1 to 15 wt. %, or 0.25 to 10 wt. %, or 0.5 to 5 wt. %, based on the total weight of the composition.

In some embodiments, the thickening agent may act as a thixotropic additive. Thixotropic additives are capable of integrating a compound that is capable of polymerizing or that has already partly or fully, polymerized, and has at least two groups available for hydrogen bonding, into a gel-like three-dimensional network. Thixotropic additives will impact a composition to have time-dependent shear thinning properties. Therefore, the oral composition may be thick or viscous under static conditions, however the composition may flow (become thinner, less viscous) over time when shaken, agitated, shear-stressed, or otherwise stressed. This property enables application of a thixotropic mixture as a semi-solid state to a body surface, which subsequently becomes substantially liquid and therefore more spreadable when applied to a surface, for example, on a tooth.

More specifically, when the composition of the present invention is sheared by squeezing the product bottle, the weak chemical bonds are broken, and a lyophobic solution is formed that can be applied to the decayed tooth. Once applied to the tooth, the particles collide, flocculation occurs, and the gel is reformed such that the dental provider can control the application.

The composition of the present invention may be thixotropic, and thus capable of undergoing an isothermal gel-sol-gel transformation. In one or more embodiments, when poured, it displays flow, but over time it reverts to being more viscous or gel like. In one or embodiments, when shear force is applied it displays flow, but overtime reverts to being more viscous or gel like. In one or more embodiments, a solid gel becomes flowable and later with time becomes solid or semi-solid. In one or more embodiments a semi-solid gel becomes flowable and later with time becomes solid or semi solid. In one or more embodiments, a liquid gel is flowable and later with time becomes solid or semi solid.

Thickening agents, which may also act as thixotropic additives may be selected from carboxymethyl cellulose, carrageenan, and silica. Thickening agents, such as xantham gum typically do not impart thixotropy properties when added to compositions.

The composition of the present invention may also include clathrates. Clathrates consist of a lattice that is capable of trapping or containing molecules. Preferably, the clathrates of the present invention comprise glyceryl acrylate and glyceryl polyacrylates, suitable for enclosing water molecules via hydrogen bonding and Van der Waals forces, such as Lubragel hydrogel (Ashland). Preferably, the clathrate of glyceryl acrylate and glyceryl polyacrylate contains about 50 wt. % of water. The clathrate may exhibit shear-thinning properties, similar to natural saliva. Another example of a clathrate suitable for the present invention is a cyclodextrin. Examples of suitable cyclodextrins include α-cyclodextrin, β-cyclodextrin, and γ-cyclodextrin, and derivatives of cyclodextrin such as methylated cyclodextrins, ethylated cyclodextrins, hydroxypropyl cyclodextrins, and hydroxyethyl cyclodextrins. The clathrates of the present invention may be present in an amount of from 0.1 wt. % to 15 wt. %, or 0.25 wt. % to 10 wt. %, or 0.5 wt. % to 5 wt. %, or 0.1 wt. % to 5 wt. %, based on the total weight of the composition.

In some embodiments, gingivitis may be treated by brushing a paste or gel containing SDF onto the teeth. In some embodiments, the paste or gel may comprise an abrasive material (i.e., aluminum hydroxide), a surfactant (i.e., sodium lauryl sulfate), and SDF that may constitute anywhere from 0.5% to 15% of the paste or gel by weight. In some embodiments, the paste or gel may also comprise fluoride and/or flavoring agent. As those skilled in the art would know, there are various ways to brush a paste or gel onto teeth. For example, a patient may first wet the brush with water, next apply a thin strip of paste or gel onto the brush, and then brush all surfaces of the teeth using short circular motions. For optimal effect, the patient may point the bristles of the brush at a 45 degree angle to the gumline. In some embodiments, the patient may let the paste or gel sit on the teeth for 1 to 5 minutes after brushing. In some embodiments, the patient may let the paste or gel sit on the teeth for extended periods of time after brushing to maximize the effect of the SDF (i.e., 5 to 30 minutes). In some embodiments, the patient may rinse the oral cavity with water after allowing the paste or gel to sit on the teeth and periodontal tissue. For example, while keeping the lips closed, the patient may make a swishing motion to move the water around the oral cavity so that it collects all the unreacted SDF. Upon completing the oral rinse, the water and SDF solution may be spit out. The patient may repeat this process as necessary.

In some embodiments, gingivitis may be treated by placing a mouthguard filled with paste or gel containing SDF onto the teeth. In some embodiments, a mouthguard may be configured to be worn on the top and bottom teeth simultaneously. For example, the mouthguard may comprise a resilient horseshoe shaped base member and molded barriers attached to the base member that form U-shaped channels above and below the base member. The top teeth may be inserted within the top channel, and the bottom teeth may be inserted within the bottom channel. In other embodiments, the mouthguard may be configured to be worn on only the top or bottom teeth at one time.

The one or more channels of the mouth guard may be filled with a paste or gel containing SDF. After the channels of the mouthguard are filled with paste or gel, the mouthguard may be placed on the teeth such that the teeth are exposed to the SDF. The mouthguard filled with paste or gel may be worn from 1 minute to up to 5 hours as the SDF treats the surface of the teeth. In some embodiments, after removing the mouthguard, the patient may rinse the oral cavity with water. For example, while keeping the lips closed, the patient may make a swishing motion to move the water around the oral cavity so that it collects all the unused SDF remaining in the oral cavity. Upon completing the oral rinse, the water and SDF solution may be spit out. The patient may repeat the rinsing process as necessary.

In some embodiments, the composition of the present invention may be prepared by mixing 38 wt. % silver diamine fluoride (aqueous) with the thickening agent, wherein the thickening agent is carboxy methyl cellulose (CMC) in an amount of from 0.1 wt. % to 15 wt. %, or 0.25 wt. % to 10 wt. %, or 0.5 wt. % to 5 wt. %, or 0.1 wt. % to 5 wt. %, based on the total weight of the composition. Enough water can be removed from the composition to compensate for the added CMC so that the final gel composition has 38 wt. % silver diamine fluoride.

In an exemplified embodiment, the composition or “gel formula” is formed by mixing an aqueous silver diamine fluoride 38 wt. % solution with carboxymethyl cellulose (CMC) as the thickening agent, wherein the final gel composition has 1.5 wt. % CMC. In another exemplified embodiment, the gel formula is formed by mixing silver diamine fluoride 38 wt. % with carboxymethyl cellulose as the thickening agent, and a clathrate of glyceryl acrylate and glyceryl polyacrylate. Preferably, the clathrate is added during a cooling phase using low-shear mixing. Enough water can be removed from the composition to compensate for all additives so that the final gel composition has 38 wt. % silver diamine fluoride.

The compositions of the present invention have a pH of no lower than 7.0, or a pH of from about 7.0 to about 13.5, or a pH of from about 7.0 to about 10.5, or a pH of from about 10 to about 10.5. The additives can themselves have varying pH while being able to withstand high pH of SDF which is 10 to 10.5.

In some embodiments, the present invention may include excipients such as nontoxic solvents which include ethanol, water, or mixtures thereof.

A coloring agent can be added to improve the appearance. A side effect of silver diamine fluoride may be the formation of silver oxide which may result in a pigmentation of the applied area. A coloring agent such as titanium dioxide, triarylmethane dye (FD&C no. 1), FD&C no. 2, FD&C no. 3, FD&C no. 4, FD&C no. 5 may be included to mask the brown color resulting from the silver oxide. Preferably, the coloring agent is triarylmethane dye. As the triarylmethane dye breaks down slowly over time, the brilliant blue dye may color shift to purple or red, indicating that it is expired.

The compositions of the present invention may also include a flavoring agent. Suitable flavoring agents may be selected from, but are not limited to, essential oils, as well as various flavoring aldehydes, esters, alcohols, and similar materials. Examples of essential oils include oils of spearmint, peppermint, wintergreen, sassafras, clove, sage, eucalyptus, marjoram, cinnamon, lemon, lime grapefruit, and orange. The flavoring agent may be included in the oral composition at concentrations of approximately 0.1 wt. % to about 5.0 wt. % by weight, or from about 0.5 wt. % to about 1.5 wt. %, based on the total weight of the oral composition.

The compositions of the present invention may also include other additives, such as mixing and stabilizing agents.

The compositions may include other active agents in addition to the SDF. For instance, the compositions may include an “antimicrobial material”, i.e., any material that, alone or in combination with other agents, kills, lyses and/or prevents the growth of microbes or otherwise reduces or eliminates the ability of a microbe to cause gingivitis and that is suitable for dental use in compositions according to the present invention. Clinical practitioners tend to use the terms “antibiotic” and “antimicrobial” synonymously. Such antimicrobial materials include compositions that comprise one or more of the following, without limitation: antibiotics (e.g., penicillin, amoxicillin, vancomycin, clindamycin, azithromycin, clarithromycin, cephalexin, cephalosporin, cephazolin, ceftriaxone, ciproflaxin, norfloxacin, rifampin, metronidazole, nitrofurantoin, tetracycline, minocycline, streptomycin, erythromycin, gentamycin, tobramycin, sulfonamides), antimicrobial peptides (e.g., defensins such as magainin, nisin), metals, metallic compounds, and metal-containing composites (e.g., sources of silver, copper, zinc, gallium, selenium, mercury, arsenic, antimony, aluminum, bismuth, gold, lead, nickel, thallium, tin, cadmium, and cobalt ions; TiO₂, Cu—TiO₂ and TiO₂ nanocomposites; Cu/SiO₂; etc.), halogens (e.g., chlorine and iodine), enzymes (e.g., lysozyme and acylase), organic cationic compounds (e.g., quaternary ammonium compounds, chlorhexidine, octenidine, cationic surfactants [BAC, CTAB, DODAB], and chitosan), organic non-cationic compounds (e.g., furanones and triclosan), and other non-organic compounds (e.g., nitric oxide). See, e.g., Cloutier et al., Trends Biotech. 33:637-652, 2015; Mittapally et al., J. Drug Delivery and Therapeutics 8:411-419, 2018; Heidenau et al., J. Materials Sci: Materials in Medicine 16:883-888, 2005).

Additional materials which may be incorporated in the compositions of the invention include whitening agents (e.g., urea peroxide, calcium peroxide, titanium dioxide, hydrogen peroxide, complexes of polyvinylpyrrolidone (PVP) and hydrogen peroxide), preservatives, vitamins (e.g., vitamin B6, vitamin B12, vitamin E, vitamin K), chlorophyll compounds, potassium salts (e.g., potassium nitrate) for the treatment of dental hypersensitivity, anti-tartar agents (e.g., sodium tripolyphosphate and di- and tetra-alkali metal pyrophosphate salts such as di- and tetrasodium pyrophosphate), remineralization agents such as calcium-based salts (e.g., dicalcium phosphate, tricalcium phosphate, precipitated calcium carbonate to be used alone or in combination with amino acids such as L-arginine), enzymes such as proteases to break down biofilms, or combinations thereof. Preferably, each of these additional materials can be present in an amount of about 0.05 wt. % to about 35 wt. %, or about 0.1 wt. % to about 10 wt. %, or about 0.1 wt. % to about 1.2 wt. % based on a total weight of the SDF composition.

### Rheology of Gel Compositions and Gel Properties

Rigidity and viscosity are two separate rheological parameters used to characterize the mechanical properties of the present invention, which may be a gel. Preferably, the compositions of the present invention possess the following properties:
1. Uniformity: the composition should be formulated so that it is and can remain uniform without separation or precipitation over time.
2. Flowability: The composition, when placed in a tube or container and expelled under shear force should be flowable.
3. Stability/Breakability: The fine balance between stability and breakability of the gel coming out of the tube or container is very delicate: on the one hand, the gel should preferably not be very runny upon release from the tube or container and not lose its thixotropy property as a result of exposure to a tooth; and on the other hand, it should be “breakable”, i.e., it should spread easily, break down and absorb onto the surface upon application of mild shear force.

### EXAMPLES

Gels can be analyzed on a RA AR-G2 rheometer using a 40 mm cone and plate rheometer, carried out at shear rates from 0.1/second to 100/second and at room temperature (about 23° C.). Gels A-D are expected to demonstrate significant thixotropic behavior as follows.

| Sample | Gel A | | Gel B | | Gel C | | Gel D | |
|---|---|---|---|---|---|---|---|---|
| Shear rate | 0.1/s | 100/s | 0.1/s | 100/s | 0.1/s | 100/s | 0.1/s | 100/s |
| Viscosity (cps) | 325,000 | 4,000 | 135,000 | 3,800 | 208,000 | 2,300 | 420,000 | 3,000 |

Fluoride and silver levels in the tooth coating composition can be measured by first diluting with water and measuring the fluoride level or silver level in the diluted sample using an ion selective probe.

The following examples were performed using liquid SDF to treat subjects having gingivitis. It is expected that the SDF gel of the present invention would provide additional advantages of control of the location of the SDF during administration. Also, the SDF gel can be covered with fluoride varnish if desired to mask the taste. SDF gel treatment can be repeated once every 3-4 months.

### Example 1

In an older patient with poor oral hygiene and gingivitis, the facial surfaces of the teeth were isolated with cotton rolls and the facial surfaces of the teeth were painted with the SDF liquid once a week for 3 weeks with a dental micro brush. Each week inflammation and bleeding decreased toward normal levels without other intervention. The SDF treatment was repeated once every 3-4 months.

### Example 2

In an adult with intellectual disability, poor oral hygiene and generalized gingivitis, hypersensitivity, and enlarged gingiva, unresolved despite dental scaling and root planing and debridement, the teeth were isolated with cotton rolls, and the buccal surfaces of the teeth were coated with the SDF liquid. After 2 minutes, the SDF was rinsed away with water. Within 3 months, dental plaque levels were reduced, and gingival health and periodontal probing depths were improved, and tooth mobility was reduced, without any further intervention. Hypersensitivity was markedly reduced. SDF treatment was repeated once every 3-4 months.

This same treatment regimen of both Examples 1 and 2 can be made with SDF gel. The teeth could be isolated with cotton rolls, and the surfaces of the teeth coated with the SDF gel. After 2 minutes, the gel is rinsed away with water. Within 3 months, dental plaque levels can be reduced, and gingival health and periodontal probing depths improved, and tooth mobility is reduced, without any further intervention. Hypersensitivity can also be markedly reduced. SDF gel treatment can be repeated once per 3-4 months.

### Example 3

In an adolescent with graft vs. host disease and gross gingivitis and rampant dental caries, traditional treatments were difficult because of the patient's fragile medical condition. SDF liquid was applied with a micro brush to the caries lesions after gentle tooth brushing, and again, applied 2 and 6 weeks later. Adjunctive chlorhexidine, fluoride gel, and xylitol chewing gum were also prescribed. Four weeks after the 6 week treatment regimen, gingival health had improved markedly. Tooth decay was arrested, and tooth sensitivity was gone.

This same treatment can be used with SDF gel. SDF gel can be applied with a micro brush to the caries lesions after gentle tooth brushing, and again, applied 2 and 6 weeks later. Adjunctive chlorhexidine, fluoride gel, and xylitol chewing gum can also be prescribed. Four weeks after the 6 week treatment regimen, gingival health can improve markedly. Tooth decay can be arrested, and tooth sensitivity can be overcome.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Other embodiments of the present disclosure will be apparent to those skilled in the art from consideration of the specification and practice of the embodiments disclosed herein. As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the disclosure being indicated by the following claims. The foregoing embodiments are susceptible to considerable variation in practice. Accordingly, the embodiments are not intended to be limited to the specific exemplifications set forth hereinabove. Rather, the foregoing embodiments are within the spirit and scope of the appended claims, including the equivalents thereof available as a matter of law.

The patentees do not intend to dedicate any disclosed embodiments to the public, and to the extent any disclosed modifications or alterations may not literally fall within the scope of the claims, they are considered to be part hereof under the doctrine of equivalents.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each range disclosed herein is to be interpreted as a disclosure of each specific value within the disclosed range that has the same number of significant digits. Thus, a range of from 1-4 is to be interpreted as an express disclosure of the values 1, 2, 3 and 4.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

1.-17. (canceled)

18. A method of preventing biofilm formation of gingivitis-causing bacteria in an oral cavity of a human subject susceptible to gingivitis comprising:
step (i): topically contacting a silver diamine fluoride composition with a surface of one or more teeth of the subject without directly contacting the gums, wherein the silver diamine fluoride composition comprises silver diamine fluoride and has at least one of the following features:
(A) the silver diamine fluoride composition is thixotropic and has a viscosity of 0.02 to 500 Pa's, as measured by a TA AR-G2 rheometer with a 40 mm cone and plate, and carried out at a temperature of about 23° C., and a shear rate of from 0.1/second to 100/second; and
(B) the silver diamine fluoride composition comprises a thickening agent, wherein the thickening agent is in an amount of 0.1 wt. % to 15 wt. %, based on a total weight of the composition;
step (ii): covering the silver diamine fluoride treated area with a fluoride varnish.

19. The method of claim 18, wherein the silver diamine fluoride is in a concentration of from 10 wt. % to 40 wt. %, based on the total weight of the composition.

20. The method of claim 18, comprising a thickening agent in an amount of 0.1 wt. % to 15 wt. %, wherein the thickening agent is a cellulose derivative; polyvinyl pyrrolidone; xanthan; an admixture of glycerin and polyacrylate; carrageenans; guar gum; gum karaya; gum arabic; gum tragacanth, or silica thickeners.

21. The method of claim 18, wherein the thickening agent is selected from carboxy methyl cellulose (CMC), microcrystalline cellulose, and an admixture of glycerin and polyacrylate.

22. The method of claim 18, wherein the silver diamine fluoride composition is prepared by mixing silver diamine fluoride with the thickening agent, wherein the silver diamine fluoride is in an amount of about 38 wt. % and the thickening agent is carboxy methyl cellulose (CMC) in an amount of 0.1 wt. % to 5 wt. % based on the total weight of the composition.

23. The method of claim 18, further comprising a clathrate of glyceryl acrylate and glyceryl polyacrylate that encloses water molecules, or a cyclodextrin.

24. The method of claim 18, further comprising a coloring agent.

25. The method of claim 24, wherein the coloring agent is triarylmethane dye.

26. The method of claim 18, further comprising a flavoring agent.

27. The method of claim 18, wherein the composition is a thixotropic fluid that can undergo an isothermal gel-sol-gel transformation.

28. The method of claim 18, wherein the composition has a pH of from 7.0 to about 13.5.

29. The method of claim 18, wherein the viscosity is from 0.1 Pa·s to 500 Pa·s, and a shear rate of from 0.1/second to 100/second; or greater than 75 Pa·s, at a shear rate of 0.1/sec, and less than 15 Pa·s, at a shear rate of 100/sec, as measured by a TA AR-G2 rheometer with a 40 mm cone and plate, and carried out at a temperature of about 23° C.

30. The method of claim 18, wherein the silver diamine fluoride provides 1,000 ppm to 75,000 ppm fluoride to the silver diamine fluoride composition, based on the total weight of the composition; or the silver diamine fluoride provides 100 ppm to 14,000 ppm silver to the silver diamine fluoride composition, based on the total weight of the composition.
